(19) United States
(12) Patent Application Publication
(10) Pub. No.: US 2022/0010299 A1
(43) Pub. Date: Jan. 13, 2022

(54) Systems and Methods to Enhance RNA Stability and Translation and Uses Thereof

(71) Applicant: The Board of Trustees of the Leland Stanford Junior University, Stanford, CA (US)

(72) Inventors: Rhiju Das, Palo Alto, CA (US); Christian A. Choe, Stanford, CA (US); Hannah K. Wayment-Steele, Stanford, CA (US); Wipapat Kladwang, Stanford, CA (US)

(73) Assignee: The Board of Trustees of the Leland Stanford Junior University, Stanford, CA (US)

(21) Appl. No.: 17/364,890

(22) Filed: Jul. 1, 2021

Related U.S. Application Data
(60) Provisional application No. 63/165,662, filed on Mar. 24, 2021.
(60) Provisional application No. 63/135,313, filed on Jan. 8, 2021.
(60) Provisional application No. 63/051,269, filed on Jul. 13, 2020.

(51) Int. Cl. C12N 15/10; C07K 14/005; A61K 31/7115

(52) U.S. Cl. CPC C12N 15/102; A61K 31/7115; C07K 14/005

(57) ABSTRACT

Embodiments herein describe systems and methods to enhance RNA translation and stability and uses thereof. Many embodiments generate RNA molecules possessing increased structure and/or reduced free energy over an initial sequence. Such RNA molecules can be used as therapeutics and/or vaccines.

### CROSS-REFERENCE TO RELATED APPLICATIONS

The current application claims priority to U.S. Provisional Patent Application No. 63/051,269, filed Jul. 13, 2020, U.S. Provisional Patent Application No. 63/165,662, filed Mar. 24, 2021, and U.S. Provisional Patent Application No. 63/135,313, filed Jan. 8, 2021; the disclosures of which are hereby incorporated by reference in their entireties.

### STATEMENT REGARDING FEDERALLY SPONSORED RESEARCH OR DEVELOPMENT

This invention was made with Governmental support under Contract Nos. GM122579, GM121487, and CA219847 awarded by the National Institutes of Health. The government has certain rights in the invention.

### FIELD OF THE INVENTION

The present invention relates to ribonucleic acid (RNA). More specifically, the present invention relates to RNA molecules with enhanced stability and translation. The present invention further relates to systems and methods to enhance RNA stability and translation by selecting for structure of RNA molecules.

### SEQUENCE LISTING

This application hereby incorporates by reference the material of the electronic Sequence Listing filed concurrently herewith. The material in the electronic Sequence Listing is submitted as a text (.txt) file entitled “06739_Seq_List_ST25.txt” created on Jun. 23, 2021, which has a file size of approximately 1.41 MB, and is herein incorporated by reference in its entirety.

### BACKGROUND

There are multiple problems with prior methodologies of effecting protein expression. For example, exogenous deoxyribonucleic acid (DNA) introduced into a cell can integrate into host cell genomic DNA at some frequency, resulting in alterations and/or damage to the host cell genomic DNA. Alternatively, the heterologous DNA introduced into a cell can be inherited by daughter cells (whether or not the heterologous DNA has integrated into the chromosome) or by offspring.

In addition, assuming proper delivery and no damage or integration of the heterologous DNA into the host genome, multiple steps must occur before the encoded protein is produced. Once inside the cell, DNA must be transported into the nucleus where it is transcribed into RNA. The RNA transcribed from DNA then enters the cytoplasm where it is translated into protein. The multiple processing steps from administered DNA to protein create lag times before the generation of the functional protein, and each step represents an opportunity for error and damage to the cell. Further, it is known to be difficult to obtain DNA expression in cells as DNA frequently enters a cell but is not expressed or not expressed at reasonable rates or concentrations. This can be a particular problem when DNA is introduced into primary cells or modified cell lines.

Attempts have been made to use RNA and messenger RNA (mRNA) as therapeutic agents. However, RNA is generally unstable and highly susceptible to degradation due to temperature, pH, and other factors.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, an RNA therapeutic includes an RNA molecule includes a 5′ untranslated region, a 3′ untranslated region, and a coding sequence, where the 5′ untranslated region is located 5′ of the coding sequence and the 3′ untranslated region is located 3′ of the coding sequence, and where the coding sequence encodes for one or more viral epitopes.

In a further embodiment, the coding sequence is selected from the group consisting of: SEQ ID NO: 5 and SEQ ID NOs: 437-439.

In another embodiment, the RNA therapeutic further includes one or more of a lubricant, a binder, a flavorant, and a coating.

In a still further embodiment, the RNA therapeutic further includes a capsule selected from a virus, a viroid, a virion, a capsid, a bacterium, a lipid nanoparticle, a micelle, a DNA structure, and an RNA structure.

In still another embodiment, at least one nucleotide in the RNA molecule is replaced with an analog selected from the group consisting of: pseudouridine, 1-methyl-pseudouridine, and 5-methyl-cytidine, 1-methoxy-pseudouridine, and pseudo-isocytidine.

In a yet further embodiment, a method for increasing RNA stability includes obtaining a target RNA sequence including a coding sequence, altering at least one nucleotide within the RNA sequence, where the altered sequence improves a metric correlated with improved RNA function, and synthesizing an RNA molecule representing the altered sequence.

In yet another embodiment, the altering step is performed by sampling a nucleotide within the target coding sequence, where the sampled nucleotide includes an unpaired nucleotide within the coding sequence, and substituting the sampled nucleotide with a new nucleotide to create a substituted coding sequence.

In a further embodiment again, the altered sequence possesses increased structure over the target coding sequence.

In another embodiment again, the metric is selected from free energy (dG) of an RNA molecule conformation, dG of the ensemble (dG(ensemble)), codon adaptation index (CAI), and expected Matthews Correlation Coefficient (MCC).

In a further additional embodiment, the metric is selected from maximum ladder distance (MLD), unpaired nucleotides, GC content, number of hairpins, number of 3-way junctions (3WJs), number of 4-way junctions, (4WJs), number of 5-way junctions (5WJs), ratios of hairpins to junctions, number of unpaired nucleotides, kissing loops, pseudoknots, tertiary contacts, multimeric designs, dimerization domains, and symmetrical structures.

In another additional embodiment, the metric is selected from mean base pair proximity, probability of unpaired nucleotides, sum of paired bases, increased structure, summed probability of being unpaired, and predicted degradation score.

In a still yet further embodiment, the substituted coding sequence possesses a lower free energy than the target coding sequence.

In still yet another embodiment, the target RNA sequence includes at least one of a poly-A tail, a 5′ untranslated region, and a 3′ untranslated region.

In a still further embodiment again, the substituting step uses a greedy GC strategy, where if a C or G substitution is possible, the nucleotide is substituted for the nucleotide.

In still another embodiment again, the altered sequence possesses a lower DegScore than the target RNA sequence, where DegScore=a*[stem nts]+b*[internal loop nts]+c*[hairpin nts]+d*[bulge nts]+e*[multiloop nts]+f*[exterior loop nts], where nts stands for nucleotides, and a-f represent coefficients for relative reactivity of nucleotides within a particular structure.

In a still further additional embodiment, the method further includes transfecting a cell with the synthesized RNA molecule.

In still another additional embodiment, the method further includes treating an individual with the synthesized RNA molecule.

In a yet further embodiment again, the synthesized RNA molecule is formulated for medical use.

In yet another embodiment again, the synthesized RNA molecule is formulated by combining the synthesized RNA molecule with at least one of a lubricant, a binder, a flavorant, and a coating.

In a yet further additional embodiment, the synthesized RNA molecule is encapsulated in at least one of a virus, a viroid, a virion, a capsid, a bacterium, a lipid nanoparticle, a micelle, a DNA structure, and an RNA structure.

In yet another additional embodiment, altering at least one nucleotide within the RNA sequence includes replacing at least one nucleotide in the RNA sequence with an analog selected from the group consisting of: pseudouridine, 1-methyl-pseudouridine, and 5-methyl-cytidine, 1-methoxy-pseudouridine, and pseudo-isocytidine.

In a further additional embodiment again, altering at least one nucleotide is iterated at least 100 times.

In another additional embodiment again, an RNA molecule to transfect a cell includes a 5′ untranslated region, a 3′ untranslated region, and a coding sequence, where the 5′ untranslated region is located 5′ of the coding sequence and the 3′ untranslated region is located 3′ of the coding sequence.

In a still yet further embodiment again, the coding sequence codes for one or more viral epitopes.

In still yet another embodiment again, the coding sequence is selected from the group consisting of: SEQ ID NO: 5 and SEQ ID NOs: 437-439.

In a still yet further additional embodiment, the coding sequence codes for green fluorescence protein.

In still yet another additional embodiment, the coding sequence is selected from the group consisting of: SEQ ID NO: 8 and SEQ ID NOs: 12-236.

In a yet further additional embodiment again, the coding sequence codes for nanoluciferase.

In yet another additional embodiment again, the coding sequence is selected from the group consisting of SEQ ID NOs: 237-436.

In a still yet further additional embodiment again, at least one nucleotide in the RNA molecule is replaced with an analog selected from the group consisting of: pseudouridine, 1-methyl-pseudouridine, and 5-methyl-cytidine, 1-methoxy-pseudouridine, and pseudo-isocytidine.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 illustrates a method to design RNA molecules with improved function in accordance with various embodiments of the invention.

FIGS. 2A-2B illustrate a generalized structures of RNA molecules in accordance with various embodiments of the invention.

FIG. 3 illustrates exemplary results of in vitro versus in vivo RNA stability in accordance with various embodiments.

FIGS. 4A-4I and 5A-5M illustrate metrics for optimized RNAs in accordance with various embodiments of the invention.

FIGS. 6A-6C illustrate energy calculations of exemplary embodiments versus benchmarking molecules in accordance with various embodiments of the invention.

FIG. 7A illustrates a structure of a target RNA molecule in accordance with various embodiments of the invention.

FIG. 7B illustrates a structure of an optimized RNA molecule in accordance with various embodiments of the invention.

FIG. 8 illustrates energy calculations of exemplary embodiments versus other methods to enhance stability in accordance with various embodiments of the invention.

FIG. 9A illustrates an exemplary embodiment of parsing of a secondary structure into categories of structural motifs of an RNA in accordance with various embodiments of the invention.

FIG. 9B illustrates chemical reactivities at individual nucleotides for an RNA construct in accordance with various embodiments of the invention.

FIG. 9C illustrates a heatmap of average reactivities for various structural motifs in accordance with various embodiments of the invention.

FIGS. 10A-10B illustrate exemplary secondary structures of RNAs in accordance with various embodiments.

FIGS. 11A-11D illustrate exemplary in vitro degradation of RNAs at various time points in accordance with various embodiments.

FIG. 12 illustrates exemplary degradation rates of RNAs possessing natural and analog substitutions in accordance with various embodiments.

FIG. 13 illustrates an exemplary secondary structure of an RNA possessing paired stems and unpaired loops in accordance with various embodiments.

FIG. 14 illustrates exemplary results of RNA degradation with single nucleotide resolution of RNAs under various conditions in accordance with various embodiments.

### DETAILED DESCRIPTION

Turning now to the drawings, systems and methods to enhance RNA stability and translation and uses thereof are provided. Many embodiments provide methods that provide an algorithmic approach to mutate an RNA sequence that optimizes stability and/or translation. In certain embodiments, the increased stability and/or translation is provided by increase in structure of the resultant RNA molecule.

There is a pressing need for vaccines against new viral pandemics like COVID-19, Ebola, flu, Zika, and other zoonotic viruses that jump from animal reservoirs into humans. mRNA molecules are considered one of the fastest ways to deploy these vaccines, but degrade and change their shape and effectiveness while stored in solution, even while refrigerated. Drug companies are not able to ship vaccines in pre-loaded syringes, making the logistical costs of deploying mass immunization currently prohibitive, and also incurring major safety risks.

A significant problem in RNA stability is self-cleavage, including from inline attack of 2′-hydroxyls on phosphates within an RNA molecule. Stabilization of RNA molecules allows for mRNA and noncoding RNA molecules to remain active and/or intact across various environments, such as pre-filled syringes, such as could be used for RNA vaccines. In a variety of embodiments, the stable RNAs will be capable of space travel, environmental/agriculture applications, dissemination in animals or the human body, which could be used in biomedicine or human performance enhancement in extreme situations.

### Methods to Improve RNA Function

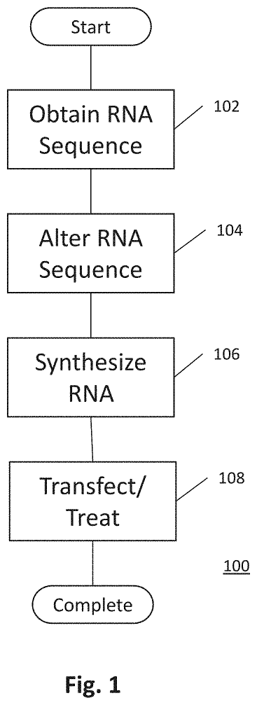

Turning to FIG. 1, a method 100 to design RNA molecules with improved function to treat and/or transfect in accordance with many embodiments is illustrated. In various embodiments, function is defined as increased stability and/or translation. Increased RNA stability includes reduced degradation of the RNA in any situation in which stability is desired, including (but not limited to) in vivo, in storage, during manufacture, or any combination thereof. At 102, many embodiments obtain an RNA sequence. In various embodiments, the RNA sequence comprises a partial or whole coding sequence, while in some embodiments, the RNA sequence comprises a coding sequence coupled with functional segments. Functional segments include (but are not limited to) a poly-A tail, a 5′ untranslated region (5′UTR), a 3′ untranslated region (3′UTR), and/or any other sequence to assist in RNA function.

At 104, many embodiments alter the RNA sequence to improve one or more elected RNA metrics. In various embodiments, the sequence alteration comprises stochastically sampling one or more nucleotides—i.e., selecting a random nucleotide in the RNA sequence. Many embodiments calculate the one or more elected RNA metrics after a sequence alteration in a sampled nucleotide and retain the new sequence, if the metric is improved in the altered sequence. In various embodiments, the nucleotide alteration does not change the resulting peptide or protein sequence.

Certain RNA metrics may predict stability and/or translation, and many embodiments elect the RNA metric from one or more of the following RNA metrics: free energy (dG) of an RNA molecule conformation, dG of the ensemble (dG(ensemble)) (e.g., an ensemble is a collection of various conformations of the same sequence), codon adaptation index (CAI), maximum ladder distance (MLD) (e.g., longest path along helices), expected Matthews Correlation Coefficient (MCC), unpaired nucleotides, number of hairpins, number of junctions (e.g., 3-way junctions (3WJs), 4-way junctions, (4WJs), 5-way junctions (5WJs), higher-order junctions), ratios of hairpins to one or more junctions, number of unpaired nucleotides in a structure, mean base pair proximity, probability of unpaired nucleotides, sum of paired bases, GC content, and other metrics that may correlate to enhance RNA stability and/or translation. In accordance with many embodiments, expected MCC is the estimated MCC of a predicted structure using the pseudo-accuracy method presented in Hamada (2010) and is a measure of how probable a predicted structure is. (See e.g., Hamada, et al., Prediction of RNA secondary structure by maximizing pseudo-expected accuracy, BMC Bioinformatics 11, 586 (2010); the disclosure of which is hereby incorporated by reference herein in its entirety.) Additionally, mean base pair proximity identifies an ensemble-averaged proximity between predicted based pairs, as calculated by equation 1, in accordance with certain embodiments.

2/N(N−1)Σᵢ^{N}Σ_{j>1}^{N}(j−i)p(i,j paired) (1)

In various embodiments, RNA stability is increased by manipulating a number of factors and/or predictors of stability. Previous methods have been developed to minimize free energy (dG) of RNA molecules. (See e.g., Zhang, et al. LinearDesign: Efficient Algorithms for Optimized mRNA Sequence Design, arxiv.org/abs/2004.10177; the disclosure of which is hereby incorporated by reference herein in its entirety.) However, free energy is but one of a number of factors that can be adjusted to increase RNA stability and/or translation.

In various embodiments, the sampling of individual nucleotides utilizes codon constraints—e.g., changes to a nucleotide are synonymous alterations, such that the resultant (or encoded) protein or peptide maintains the same amino acid sequence. Further embodiments include a “greedy GC” strategy—e.g., a strategy where G or C substitutions are preferred, such as (for example) a G or C substitution in the third spot of a codon trinucleotide. For example, the codon UCU could be altered to UCC or UCG, rather than UCA, while still encoding for serine, thus increasing GC content. Additionally, greedy GC or GC preferred strategies can be used outside of coding regions and codons, such as UTRs (e.g., 5′UTRs and 3′UTRs) and any other non-coding feature in an RNA molecule that can be changed without altering the function of the feature.

Additionally, various embodiments utilize the probability that certain bases are unpaired in the RNA's secondary structure. Some of these embodiments utilize a summed probability of being unpaired (Sum p(unp)), which is a count of the average number of nucleotides in the RNA that are expected to be unpaired. This determination can be computed in various RNA modeling packages. Certain embodiments use an RNA modeling package selected from Vienna 2, RNAstructure, CONTRAfold, and EternaFold to calculate probability of base paring and energy of various structural states of the RNA sequence. The Sum p(unp) metric provides an estimate of relative degradation rates of different mRNAs. In various embodiments, Sum p(unp) makes one or more assumptions selected from (1) the statistical mechanical ensemble of secondary structures predicted by the RNA modeling package reflects the RNA's actual ensemble in the experimental conditions, and (2) the rate of degradation at a given nucleotide is 0.0 if the nucleotide is base paired (in a helix), and some constant rate if it is unpaired. In certain embodiments, Sum p(unp) is multiplied by a constant chemical degradation rate to be turned into an overall rate of degradation for a full-length RNA. However, in comparisons between RNA molecules, the multiplication factor can be ignored.

In many embodiments, the relation of Sum p(unp) to degradation rate can be shown mathematically. The probability of the full length RNA remaining undegraded after time t drops exponentially as equation 2:

exp(−k_TOT t) (2)

which should equal the product of probabilities of each nucleotide remaining undegraded, exp(−k_1 t)*exp(−k_2 t)*exp(−k_N t), where k_i is the rate of each nucleotide i from 1 to the number of nucleotides N, and assumed to be proportional to the fraction of time the nucleotide I is unpaired, p_i(unp). Therefore, k_TOT is the sum of k_i and is proportional to Sum p(unp).

In various embodiments, altering the RNA sequence 104 is performed iteratively to improve the one or more elected RNA metrics. In some embodiments, altering the RNA sequence 104 is iterated at least 100 times, at least 250 times, at least 500 times, at least 750 times, at least 1000 times, at least 1500 times, at least 2000 times, at least 2500 times, at least 3000 times, at least 3500 times, at least 4000 times, at least 4500 times, at least 5000 times, at least 7500 times, at least 10,000 times, or more.

At 106, many embodiments synthesize an RNA construct representing the designed RNA sequence. Various embodiments chemically and/or biochemically synthesize the RNA construct via various known technologies. Example methods of synthesis include phosphoramidite chemistry, T7 polymerase, and any other known or applicable means of synthesizing an RNA construct or oligonucleotide. In various embodiments, the synthesized oligonucleotide comprises the coding sequence, after which, additional features (e.g., cap moiety, UTRs, etc.) can optionally be ligated to the coding sequence. In certain embodiments, the synthesized oligonucleotide comprises a full-length construct, including a cap moiety, 5′UTR, coding sequence, 3′UTR, tailing sequence or poly-A tail, and any other feature of interest to include within the construct.

Certain embodiments synthesize the construct using RNA nucleotides, while some embodiments synthesize the construct using DNA nucleotides, and additional embodiments synthesize the construct using a combination of RNA and DNA nucleotides. Further, some embodiments synthesize the oligonucleotide and its complement, which can be paired together to form a double stranded molecule, and some embodiments synthesis the oligonucleotide such that portions of the molecule are double-stranded and other portions of the molecule are single-stranded. Certain embodiments incorporate nucleotide analogs into the synthesized oligonucleotides, including pseudouridine, inosine, 5-methyl-cytosine, and other known analogs.

Optionally at 108 of some embodiments, an RNA construct is transfected into a cell and/or used in a treatment of a subject. As noted elsewhere herein, RNA constructs can have many purposes, reporter gene expression, vaccines, other RNAs for translation (such as for gene therapy, protein production, or any other use of protein production), and functional RNAs (e.g., small RNAs, interfering RNAs, ribosomal RNAs, and any other functional RNAs). As such, transfecting a cell in accordance with certain embodiments inserts the RNA into a cell directly, such as through microinjection, particle bombardment, electroporation, heat shock, or other direct transfection methods. In certain embodiments involving the treatment of an individual, an RNA construct can be formulated for a medical use, including by combining it with one or more buffers, lubricants, binders, flavorants, and coatings. Various embodiments encapsulate the RNA construct for transfection, such as through a virus (e.g., adeno-associated viruses (AAVs)), viroids, virions, capsids, bacteria (e.g., Agrobacterium spp.), lipid nanoparticles, micelles, and/or larger DNA and/or RNA structures suitable for targeting and/or stability, and/or other methods of encapsulating an RNA for transfection.

### RNA Constructs

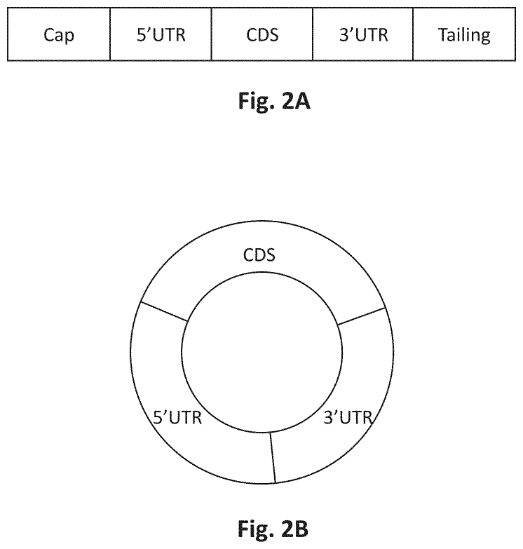

Turning to FIGS. 2A-2B, many embodiments are directed to RNA molecules for use as a therapeutic, vaccine, and/or to produce a protein or peptide of interest. FIG. 2A illustrates a general diagram of linear RNA molecules in accordance with various embodiments, while FIG. 2B illustrates a general diagram of a circular RNA molecule in accordance with some embodiments.

Additional embodiments possess a 5′ untranslated region (5′UTR) sequence and/or a 3′UTR sequence. Certain embodiments place the 5′UTR near the 5′ end of the RNA molecule (e.g., upstream a coding or functional sequence), while the 3′UTR is located near the 3′ end of the molecule (e.g., downstream a coding or functional sequence). In some embodiments, the 5′UTR is located at the 3′ end of the cap, while additional embodiments utilize a 5′UTR without a cap sequence. Similarly, a 3′UTR can be placed at the 3′ end of a molecule. Certain embodiments select a 5′UTR and/or a 3′UTR for a variety of factors to increase stability and/or translation based on an innate sequence, while others select a 5′UTR and/or a 3′UTR for that may pose improved translation and/or stability based on a particular coding sequence of interest. Many possible 5′UTRs and 3′UTRs are known in the art, which are used in various embodiments. Some specific embodiments select the 5′UTR from human hemoglobin beta subunit (HBB) (SEQ ID NO: 1). Additional embodiments select the 3′UTR from HBB (SEQ ID NO: 2).

Many embodiments possess a coding sequence (CDS) located 3′ from the 5′UTR, and/or 5′ of the 3′UTR. In many embodiments, the beginning of the CDS is marked with the start codon AUG. In many embodiments, the end of the CDS is marked with a stop codon. The coding sequence is a designed sequence of interest to encode a protein or peptide of interest. In certain embodiments, the coding sequence encodes an epitope or other antigen to induce an immune response, thus allowing for use as a vaccine. In various embodiments, the protein or peptide of interest is used as a therapeutic, such that the protein or peptide of interest replaces or supplements a dysfunctional protein or peptide. In some embodiments, the protein or peptide of interest corrects for dysfunction of another protein or peptide. While protein coding sequences are described in the context of this exemplary embodiment, additional embodiments possess other functional sequences for non-coding RNAs, such as RNAs that guide genome editing (e.g., gRNA for use in CRISPR system) and/or coat chromatin.

Certain linear embodiments possess a 5′ cap moiety. Some embodiments utilize a 7-methyl guanosine triphosphate as the cap moiety, but various additional cap sequences are known in the art for a 5′ cap moiety. Additional embodiments possess a cap-proximal sequence for an mRNA located at the 5′ end of the mRNA. Various cap sequences are known in the art for a 5′ cap-proximal sequence. Certain embodiments use a small triplet, such GGG as the cap-proximal sequence.

Additionally, some linear embodiments possess a tailing sequence located at the 3′ end of a molecule (e.g., 3′ of the 3′UTR). In various embodiments the tailing sequence is used to add a poly-A tail or other structural sequence to an RNA molecule. In some embodiments, the tailing sequence is selected as SEQ ID NO: 3.

Further embodiments include additional sequences or components that can be used to identify sequences and/or to increase translatability, to increase stability, or to any other characteristic that may be beneficial for an RNA molecule.

### RNAs Incorporating Nucleotide Analogs

As noted above, numerous embodiments incorporate one or more nucleotide analogs. Such embodiments incorporating nucleotide analogs possess increased stability and/or translation over RNA molecules possessing solely natural (e.g., A, C, G, U) nucleotides. Additional embodiments incorporate one or more nucleotide analogs to replace some or all of the natural nucleotides within an RNA sequence. For example, some embodiments replace 5%, 10%, 15%, 20%, 25%, 30%, 35%, 40%, 45%, 50%, 55%, 60%, 65%, 70%, 75%, 80%, 85%, 90%, 95%, or 100% of a natural nucleotide with an analog (e.g., replace uracil with pseudouridine, replace cytidine with 5-methyl-cytidine, etc.). Further embodiments incorporate nucleotide analogs along with additional sequence alterations, including (but not limited to) sequence alterations for codon optimization, increased structure, or any other sequence alteration.

Pseudouridine, 1-methyl-pseudouridine, and 5-methyl-cytidine provide accurate mRNA translation in human cells, and may even enhance translation and in vivo stability and favorably reduce undesired innate immune response. (See, e.g., Karikó K, Muramatsu H, Welsh F A, et al. Incorporation of pseudouridine into mRNA yields superior nonimmunogenic vector with increased translational capacity and biological stability. Mol Ther. 2008; 16(11):1833-1840. doi:10.1038/mt.2008.200; U.S. Pat. No. 8,278,036 B2; and David M. Mauger, B. Joseph Cabral, Vladimir Presnyak, Stephen V. Su, David W. Reid, Brooke Goodman, Kristian Link, Nikhil Khatwani, John Reynders, Melissa J. Moore, lain J. McFadyen PNAS November 2019, 116 (48) 24075-24083; DOI: 10.1073/pnas.1908052116; the disclosures of which are hereby incorporated by reference in their entireties.)

However, in vivo and in vitro stability are two independent problems for RNA. In vivo stability can depend on untranslated sequences at 3′-ends of mRNAs, structures and sequences that signal decay, process that identify premature stop codons, RNA elements recognized by cellular endonucleases and exonucleases, and ribosome-dependent decay processes. (See, e.g., Koh, W. S., Porter, J. R. & Batchelor, E. Tuning of mRNA stability through altering 3′-UTR sequences generates distinct output expression in a synthetic circuit driven by p53 oscillations. Sci Rep 9, 5976 (2019). doi: 10.1038/s41598-019-42509-y; Park E, Maquat L E. Staufen-mediated mRNA decay. Wiley Interdiscip Rev RNA. 2013 Jul.-Aug.; 4(4):423-35. doi: 10.1002/wrna.1168. Epub 2013 May 16. PMID: 23681777; PMCID: PMC3711692; Brogna, S., Wen, J. Nonsense-mediated mRNA decay (NMD) mechanisms. Nat Struct Mol Biol 16, 107-113 (2009). doi: 10.1038/nsmb.1550; Blandine C. Mercier, Emmanuel Labaronne, David Cluet, Alicia Bicknell, Antoine Corbin, Laura Guiguettaz, Fabien Aube, Laurent Modolo, Didier Auboeuf, Melissa J. Moore, Emiliano P. Ricci bioRxiv 2020.10.16.341222; doi: 10.1101/2020.10.16.341222; the disclosures of which are hereby incorporated by reference in their entireties.) RNA degradation in aqueous buffers can occur in much longer time scales, but this can accelerate in the presence of magnesium (Mg²+) or in high pH. (See e.g., Hannah K. Wayment-Steele, Do Soon Kim, Christian A. Choe, John J. Nicol, Roger Wellington-Oguri, R. Andres Parra Sperberg, Po-Ssu Huang, Eterna Participants, Rhiju Das bioRxiv 2020.08.22.262931; doi: 10.1101/2020.08.22.262931; the disclosure of which is hereby incorporated by reference in its entirety.) Common strategies to stabilize mRNAs for in vivo stability (including appending long poly adenosine stretches; >100 As) can actually destabilize RNAs in vitro by adding additional locations for possible hydrolysis. Additionally, embedded structured segments, which are expected to stabilize RNAs against in-line hydrolysis have been shown to decrease stability of mRNA's inside human cells through a process termed structure-mediated RNA decay (SRD), involving cellular factors UPF1 and G3BP1. (See e.g., Fischer, Joseph W. et al. Molecular Cell, Volume 78, Issue 1, 70-84.e6; the disclosure of which is hereby incorporated by reference in its entirety.)

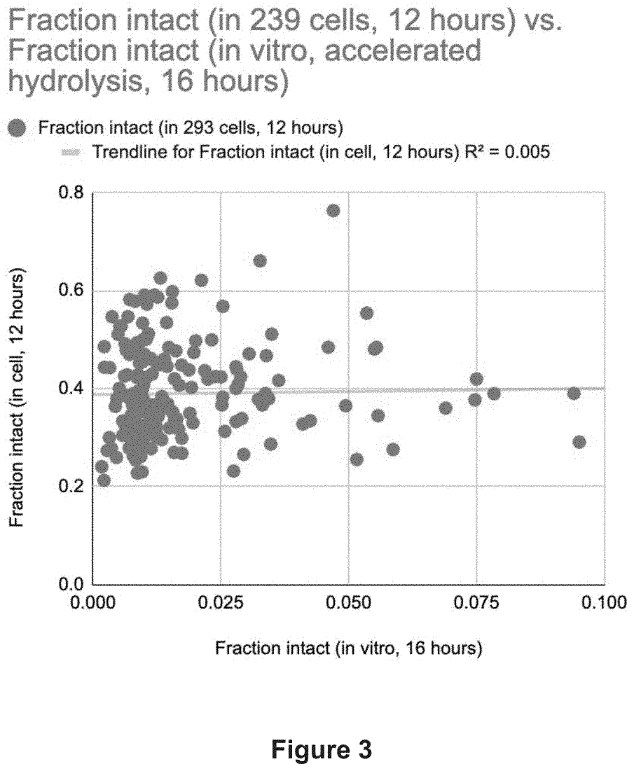

Turning to FIG. 3, exemplary results of an empirical study of an mRNA library coding for nanoluciferase show that decay rates in human cells exhibit no correlation with in vitro decay rates. In FIG. 3, the in cell and in vitro stability possess an r² value of 0.0005, indicating no correlation. Such measurements were carried out using a library of 233 mRNAs of varying lengths (507-1215 nucleotides) and sequences. The measurements involve a reverse-transcription based assay to count RNAs remaining after degradation times, with strong reproducibility in ranking mRNA stabilities between time points or in replicates. In-cell measurements involved mRNAs transfected into human 293 cells. In vitro measurements were carried out under hydrolysis conditions (10 mM MgCl₂, 50 mM Na-CHES, pH 10.0, 24° C.) that accelerate hydrolysis by ˜100× compared to neutral buffers without Mg²⁺.

Analogs like pseudouridine have been proposed to lead to enhanced mRNA stability in cells by stabilizing Watson-Crick base-paired helices which somehow prevent ribosome collisions and to decrease recognition by in-cell RNA sensors (e.g., in innate immunity pathways). (See e.g., David M. Mauger, et al.; cited above.) However, such effects have no applicability in in vitro environments, where immunity pathways and ribosomes do not exist. Instead, analogs may change neutrophilicity of the nucleoside's 2′-hydroxyl group, which is the attacking group in the chemical reaction, or analogs may enhance base stacking creating a local structural effect. (See e.g., Yingfu Li and Ronald R. Breaker Journal of the American Chemical Society 1999 121 (23), 5364-5372 DOI: 10.1021/ja990592p; and Davis D R. Stabilization of RNA stacking by pseudouridine. Nucleic Acids Res. 1995 Dec. 25; 23(24):5020-6. doi: 10.1093/nar/23.24.5020. PMID: 8559660; PMCID: PMC307508; the disclosures of which are hereby incorporated by reference in their entireties.) Neither the neutrophilicity or local structural effect is related to the Watson-Crick base pairing or changed recognition by proteins proposed for in-cell effects of an analog. Thus, it would not be obvious or trivial to introduce nucleotide analogs into an RNA molecule to increase in vitro stability of an RNA molecule.

Many embodiments are directed to RNA molecules comprising at least one nucleotide substitution. In many of these embodiments, the nucleotide substitution is a substitution of a natural nucleotide (e.g., A, C, G, U) with an analog and/or chemically modified analog. Such analogs include (but are not limited to) pseudouridine, 1-methyl-pseudouridine, and 5-methyl-cytidine, 1-methoxy-pseudouridine, pseudo-isocytidine, and/or any other nucleotide analog. Many embodiments are directed to methods to improve in vitro stability of an RNA molecule by incorporating one or more of the nucleotide analogs into the RNA molecule.

### Coding Sequences

As noted elsewhere herein, many embodiments select coding sequences to produce a protein or peptide of interest. Proteins and/or peptides of interest can be used for a therapeutic effect, including to generate an immunogenic response by producing an epitope, antigen, or other immunogenic molecule. While some proteins and/or peptides of interest can be used for cellular signaling and/or isolation. The number of possible sequences that code for a given amino acid sequence is astronomically large (greater than 10\hat{ }50) so it is not possible to synthesize all of them and test them. Design principles are needed to select a subset of this large set of sequences for experimental characterization.

As illustrative examples of some embodiments, certain embodiments are directed to an antigenic epitope, such as SEQ ID NO: 4, to design an RNA vaccine. The epitope (SEQ ID NO: 4) possesses a coding sequence of SEQ ID NO: 5. However, because numerous codons within a coding sequence can be synonymously mutated to result in the same peptide (e.g., SEQ ID NO: 4), a coding sequence can be relaxed to possess IUPAC constraints revealed in SEQ ID NO: 6.

Additionally, entire proteins can be created by some embodiments. As an illustrative example, SEQ ID NO: 7 includes the peptide sequence for green fluorescence protein (GFP). Additionally, SEQ ID NO: 8 includes a coding sequence for GFP, and SEQ ID NO: 9 includes a coding sequence with IUPAC constraints for GFP. Further embodiments possess a coding sequence for GFP selected from SEQ ID NOs: 12-236 and SEQ ID NOs: 440-1158.

Further embodiments include coding sequences directed to a luciferase, such as a nanoluciferase. In some of these embodiments, the nanoluciferase coding sequence is selected from SEQ ID NOs: 237-436.

As noted above, certain embodiments, are directed to immunogenic coding sequences. Some of these embodiments are directed to a multi-epitome vaccine (MEV) coding sequence. In various embodiments, the MEV is specific for a coronavirus, such as SARS-CoV-2, the virus that causes Covid-19. In certain embodiments, the coronavirus specific MEV is selected from SEQ ID NOs: 437-437 and SEQ ID NOs: 1159-1164.

### Characteristics of Molecules

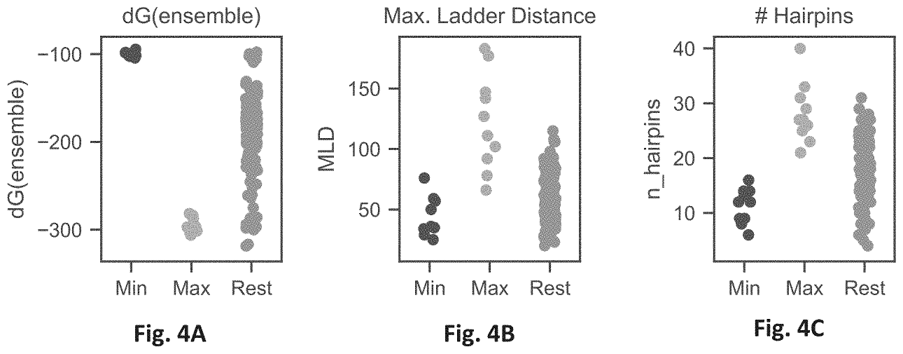

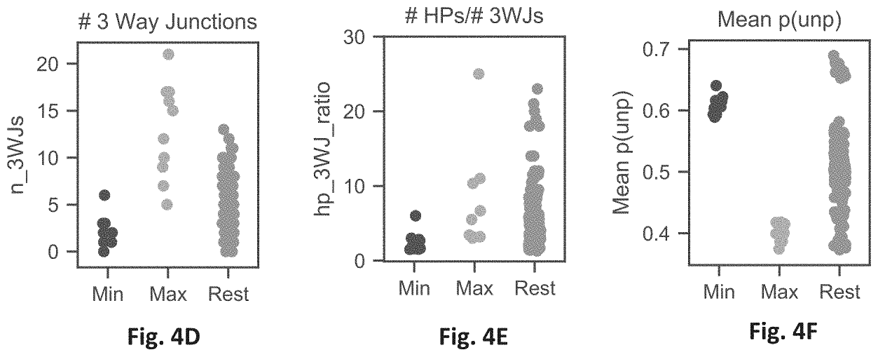

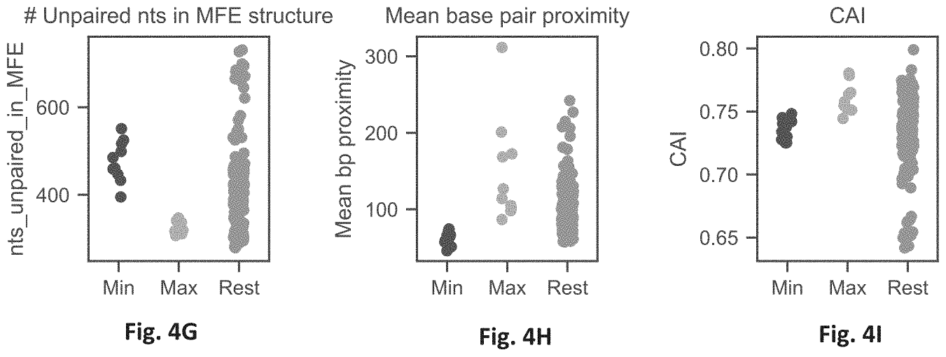

Turning to FIGS. 4A-4I, various metrics are plotted for RNAs optimized for a particular parameter. In particular, FIG. 4A illustrates results for exemplary embodiments minimizing (Min) and maximizing (Max) dG(ensemble) as well as the dG(ensemble) for embodiments optimized for other parameters (Rest). Similarly, FIG. 4B illustrates results for exemplary embodiments optimized for Maximum Ladder Distance (MLD); FIG. 4C illustrates results for exemplary embodiments optimized for the number of hairpins; FIG. 4D illustrates results for exemplary embodiments optimized for the number of 3-Way Junctions; FIG. 4E illustrates results for exemplary embodiments optimized for a ratio of hairpins to 3-Way Junctions; FIG. 4F illustrates results for exemplary embodiments optimized for Mean p(unp); FIG. 4G illustrates results for exemplary embodiments optimized for the number of unpaired nucleotides in a minimum free energy (MFE) structure; FIG. 4H illustrates results for exemplary embodiments optimized for Mean Base Pair Proximity; and FIG. 4I illustrates results for exemplary embodiments optimized for CAI.

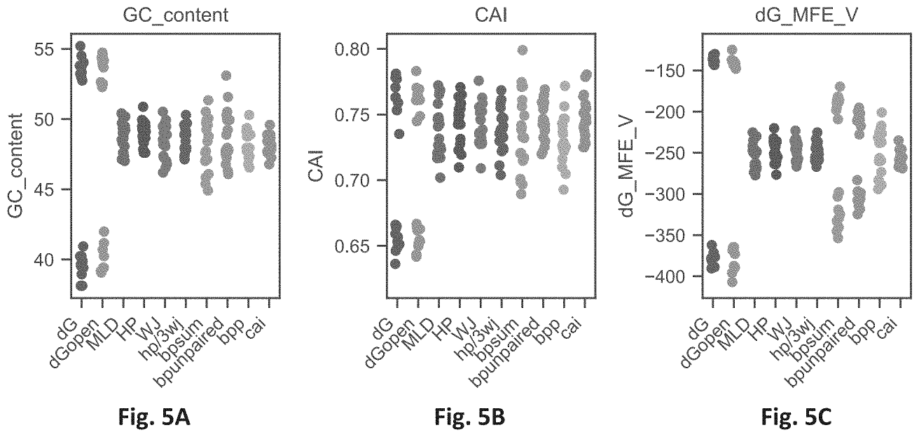

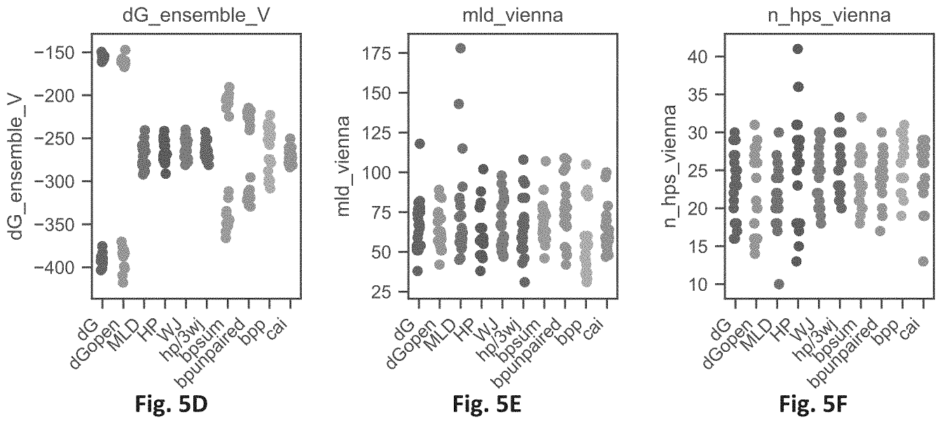

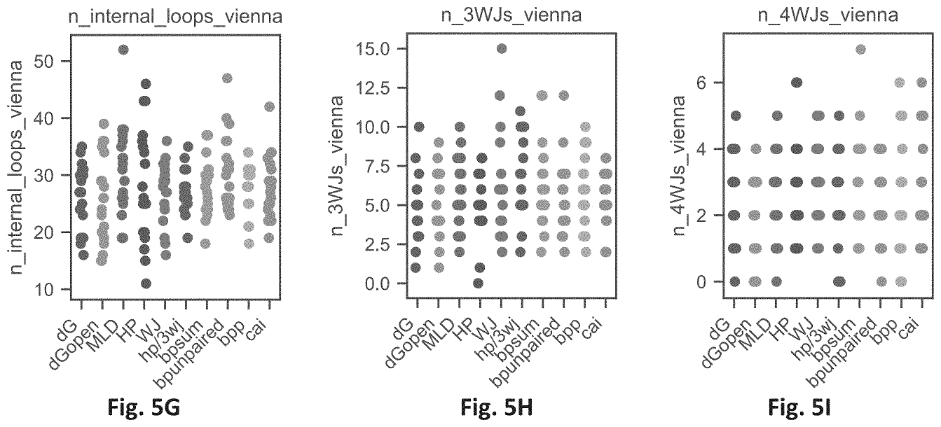

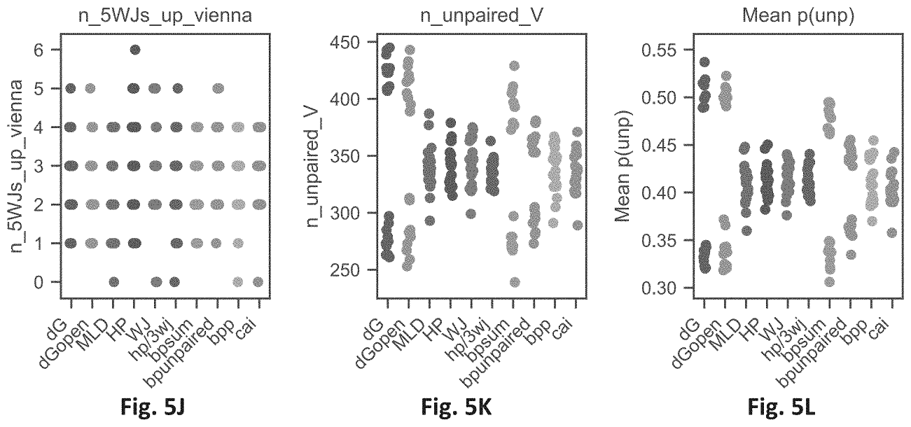

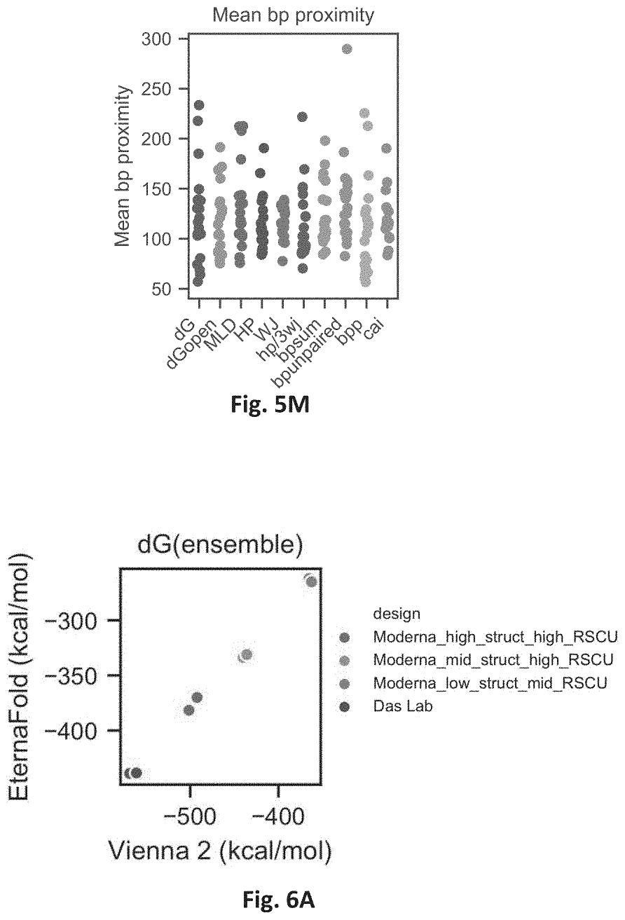

Similarly, FIGS. 5A-5M illustrate results from exemplary embodiments showing metrics, including GC content (FIG. 5A), CAI (FIG. 5B), dG of MFE (FIG. 5C), dG(ensemble) (FIG. 5D), Maximum Ladder Distance (MLD) (FIG. 5E), number of hairpins (FIG. 5F), number of internal loops (FIG. 5G), number of 3-Way Junctions (FIG. 5H), number of 4-Way Junctions (FIG. 5I), number of 5-Way Junctions (FIG. 5J), number of unpaired nucleotides (FIG. 5K), Mean p(unp) (FIG. 5L), and Mean base pair proximity (FIG. 5M) for embodiments optimized for the various conditions listed on the X-axis, including dG, dGopen, MLD, number of hairpins (HP), number of junctions (WJ), ratio of hairpins to junctions (hp/3wj), sum of paired bases (bpsum), number of unpaired bases (bpunpaired), base pair proximity (bpp), and CAI.

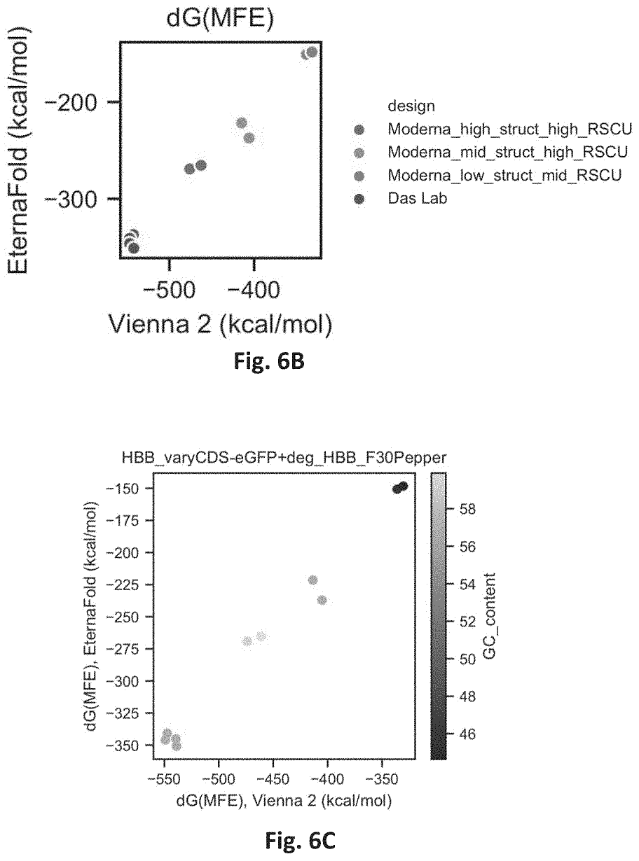

Turning to FIGS. 6A-6C, free energy calculations based on EternaFold and Vienna 2 are plotted of certain exemplary embodiments. As illustrated, various embodiments possess lower free energy as determined of ensemble (FIG. 6A) and minimal free energy (MFE) (FIG. 6B) as compared to various benchmarking RNAs possessing high levels of structure, middle levels of structure, and low levels of structure. In all instances, exemplary embodiments possess approximately a 25% reduction in free energy than the benchmarking proteins. Additionally, FIG. 6C illustrates that the exemplary embodiments possess increased levels of GC content than the low and middle levels of structure, however these exemplary embodiments possessed slightly lower GC content than the high structure benchmarking proteins (˜56% vs. ˜59% GC).

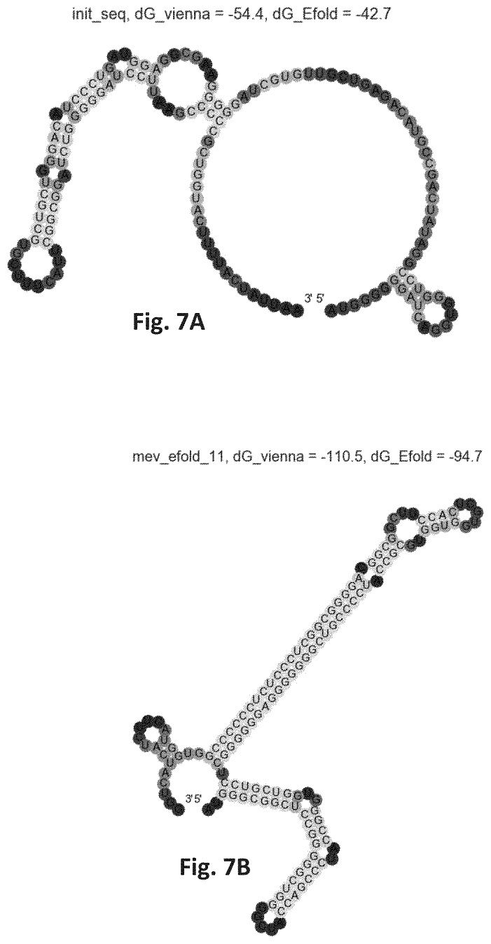

Turning to FIGS. 7A-7B, structures of a starting molecule (FIG. 7A) and an exemplary molecule (FIG. 7B) are illustrated. In particular, FIG. 7A illustrates a starting sequence (SEQ ID NO: 10), while FIG. 7B illustrates and exemplary embodiment (SEQ ID NO: 11) that has been optimized for lower free energy (dG) and structure based on the starting sequence. The darker shading in FIGS. 7A-7B demonstrate a higher probability of unpaired nucleotides, while lighter shading indicates a higher probability of paired nucleotides. As seen in FIGS. 7A-7B, optimized molecules of various embodiments possess increased structure and lower free energy.

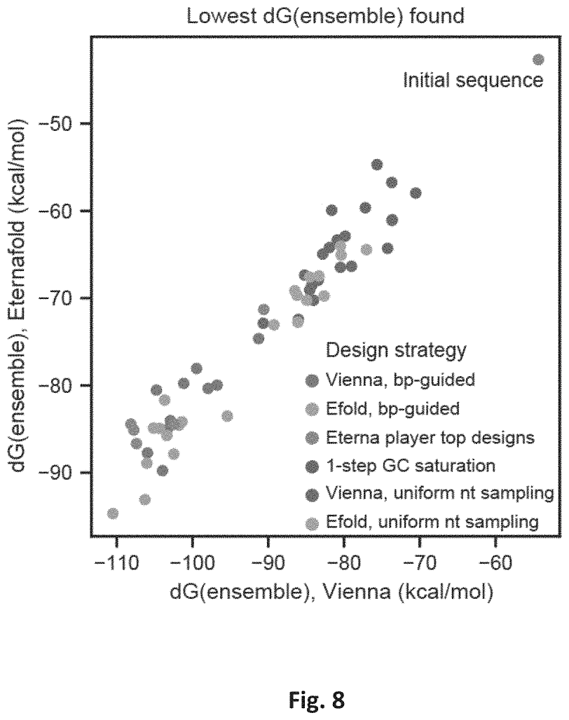

Turning to FIG. 8, free energy as determined from Vienna and EternaFold are plotted for various methods to optimize molecules. As illustrated, various exemplary embodiments generally possess lower predicted free energy than other methods to design or optimize RNA molecules (e.g., Vienna, GC saturation, etc.)

### Determination of RNA Stability

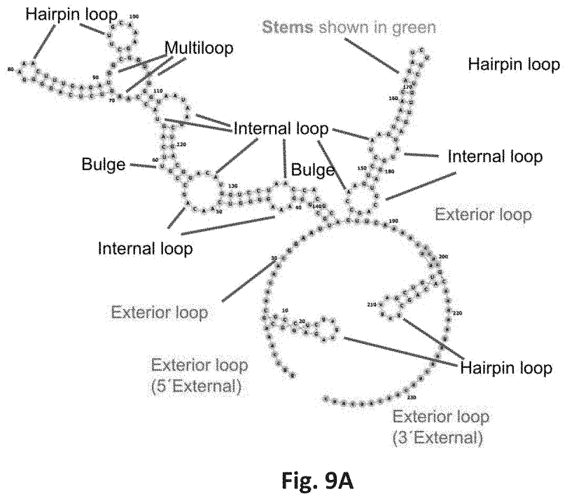

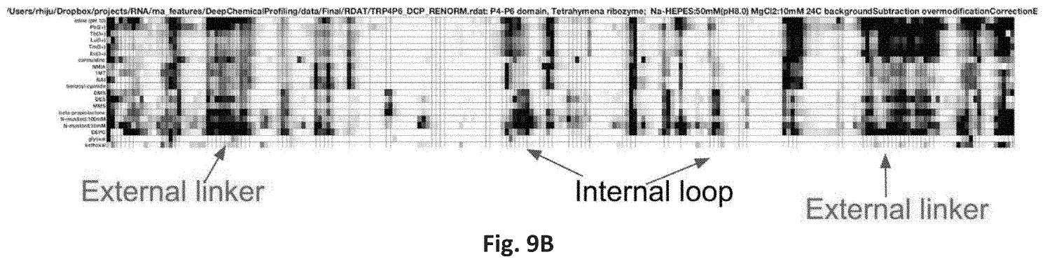

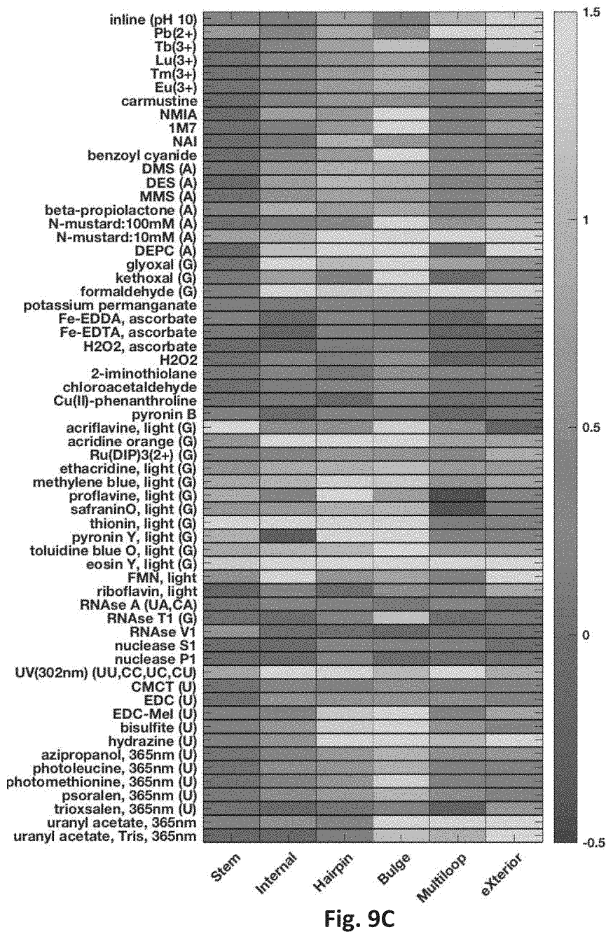

Turning to FIGS. 9A-9C, an exemplary embodiment to determine RNA stability is illustrated. In particular, FIG. 9A illustrates an exemplary embodiment of parsing of a secondary structure into categories of structural motifs of a P4-P6 domain of the Tetrahymena ribozyme with two flanking GAGUA hairpins. As illustrated in FIG. 9A, structural features can generally include stem, interior loop, hairpin loop, bulge, multiloop, and exterior loop. However, some of these structures can be subdivided further, such that exterior loops can include linker loops and external loops, while multiloops can be divided into multi-way junctions (e.g., 3-way, 4-way, etc.). By identifying the reactivity of various structures, degradation scores are determined for various molecules, in accordance with many embodiments.

FIG. 9B illustrates chemical reactivities at each nucleotide (x-axis) with respect to different chemical modifiers (y-axis), applied to the P4-P6 domain of a Tetrahymena ribozyme (e.g., FIG. 7A). In FIG. 9B, regions expected to be unpaired for this RNA are marked with vertical lines. Of these regions, external linkers (marked with red labels) show consistently high reactivity (dark colors), indicating how embodiments are capable of identifying regions with higher rates of degradation under certain conditions.

Additionally, FIG. 9C illustrates a heatmap of average reactivities for various structural motifs (x-axis) with respect to 61 different chemical modifiers. In this exemplary embodiment, the heatmap is normalized to mean reactivities and median-centered before taking the averages.

Based on nucleotide reactivity, a degradation score can be determined and/or predicted for a particular sequence based on the predicted structure for an RNA sequence. The following equation provides one formula for calculating a degradation score (DegScore), in accordance with some embodiments:

DegScore=a*[stem nts]+b*[internal loop nts]+c*[hairpin nts]+d*[bulge nts]+e*[multiloop nts]+f*[exterior loop nts],

Where nts stands for nucleotides, and
a-f represent coefficients for relative reactivity of nucleotides within a particular structure. In many embodiments, the coefficients range from 0.0-1.0 (e.g., if nucleotides in exterior loops are 5× more reactive than nucleotides in an internal loop, coefficient b could equal 0.2, while coefficient f could equal 1.0).

### Exemplary Embodiments

Although the following embodiments provide details on certain embodiments of the inventions, it should be understood that these are only exemplary in nature, and are not intended to limit the scope of the invention.

### Example 1: Incorporating Nucleotide Analogs

Background: Current mRNA therapeutics and vaccine efforts that have focused explicitly on increasing stability of mRNAs and reducing costs of manufacturing (e.g., with self-amplifying mRNA vectors) have not explored use of chemical modifications for stabilization, despite widespread know-how for incorporating chemical modified nucleotides during transcription. (See e.g., Zhang N N, Li X F, Deng Y Q, et al. A Thermostable mRNA Vaccine against COVID-19. Cell. 2020; 182(5):1271-1283.e16. doi:10.1016/j.cell.2020.07.024; McKay, P. F., Hu, K., Blakney, A. K. et al. Self-amplifying RNA SARS-CoV-2 lipid nanoparticle vaccine candidate induces high neutralizing antibody titers in mice. Nat Commun 11, 3523 (2020). doi: 10.1038/s41467-020-17409-9; Erasmus, J. H., et al. Science Translational Medicine 5 Aug. 2020: Vol. 12, Issue 555, eabc9396 DOI: 10.1126/scitranslmed.abc9396; the disclosures of which are hereby incorporated by reference in their entireties.)

Methods: A nanoluciferase sequence was modified to include additional structure (e.g., stronger base pairing regions) and/or to incorporate nucleotide analogs.

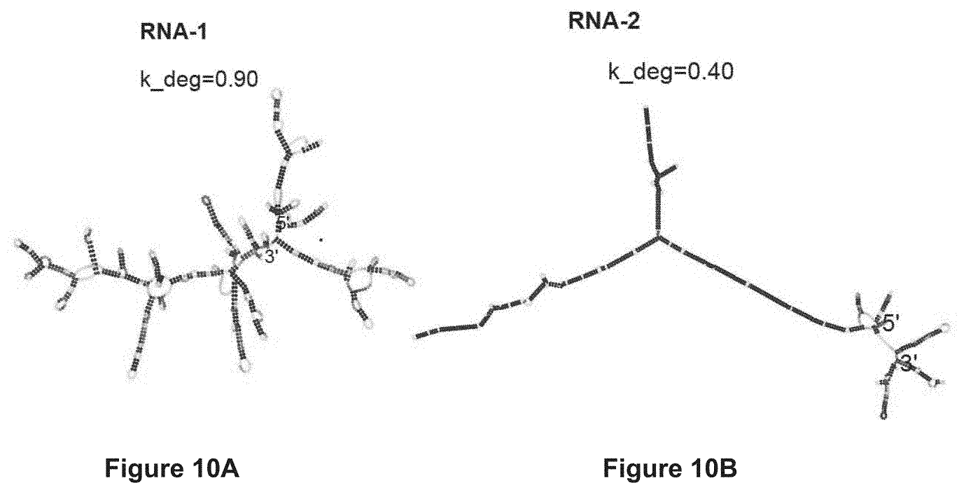

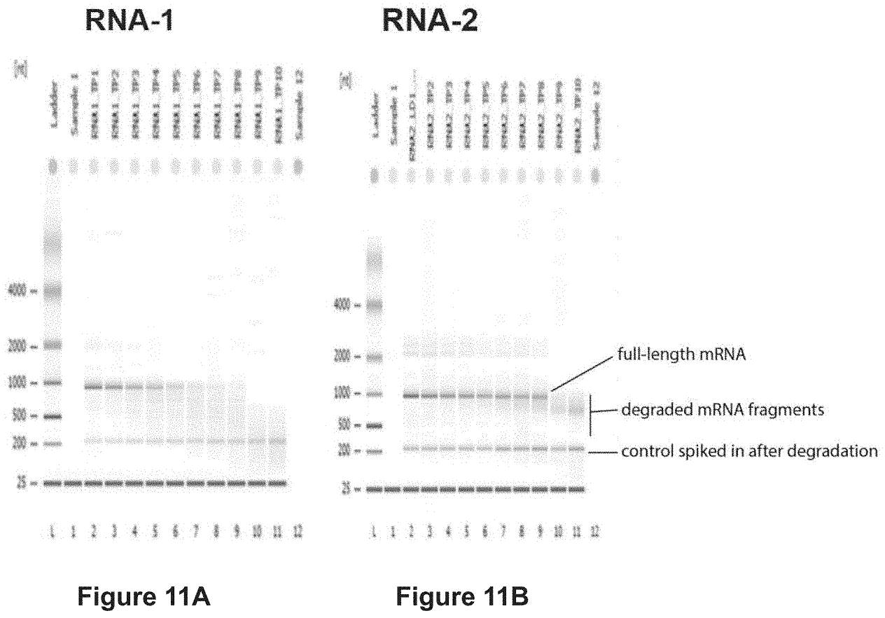

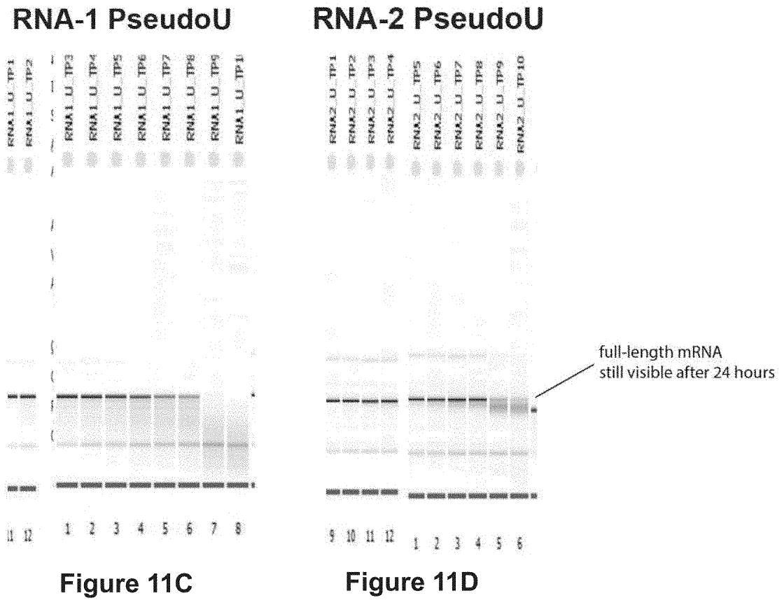

Results: Turning to FIGS. 10A-10B, exemplary RNAs used in testing are illustrated encoding for nanoluciferase. FIG. 10A illustrates the secondary structure of RNA-1 (SEQ ID NO: 1165) possessing short, weakly stems, while FIG. 10B illustrates the secondary structure of RNA-2 (SEQ ID NO: 1166) possessing longer and stronger pairing regions. The stabilities of these RNAs over time are illustrated as electropherograms in FIGS. 11A-11D, specifically at time points of 0 h, 0.5 h, 1 h, 1.5 h, 2 h, 3 h, 4 h, 5 h, 18 h, and 24 h. Specifically, FIG. 11A illustrates the in vitro stability of the SEQ ID NO: 1165, while FIG. 11B illustrates the in vitro stability of the SEQ ID NO: 1166. FIGS. 11A-11B illustrate that while stronger secondary structures of SEQ ID NO: 1166 provide some increased stability, both RNAs (SEQ ID NOs: 1165-1166) show some degradation immediately leading to eventual, full degradation. FIGS. 11C-11D illustrate stability of (SEQ ID NOs: 1165-1166), wherein the natural uridines have been substituted with pseudouridine. As illustrated in FIGS. 11C-11D, the integration of pseudouridine increases stability in both RNAs over time, and full-length RNA is still present in the higher structured SEQ ID NO: 1166 after 24 hours. FIGS. 11A-11D further possess a control RNA spike in (SEQ ID NO: 1171) applied after degradation.

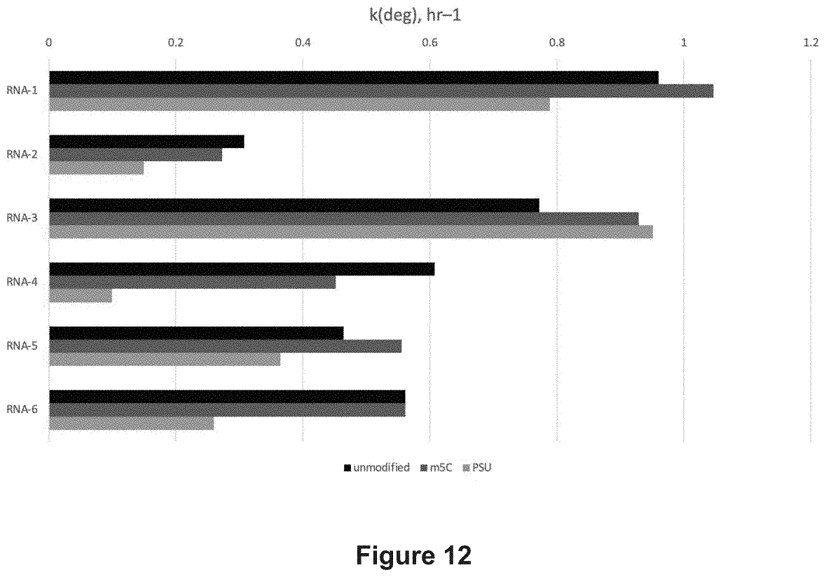

Turning to FIG. 12, degradation rates of various RNAs (RNAs 1-6; SEQ ID NOs: 1165-1170, respectively) possessing 5-methyl-cytosine (m5C) or pseudouridine (PSU) substitutions. For 5 of the 6 RNAs, incorporation of pseudouridine (PSU) reduces degradation rates; in three cases, the improvement is more than 2-fold. Interestingly, the cases showing strongest effects are the RNAs that were designed to have the most structure; thus, the use of pseudouridine is synergistic with other design strategies to stabilize mRNA against in vitro degradation by hydrolysis. Supporting the specificity of the analog-substitution concept, another modification, 5-methyl-cytidine (a C analog) did not change degradation rates.

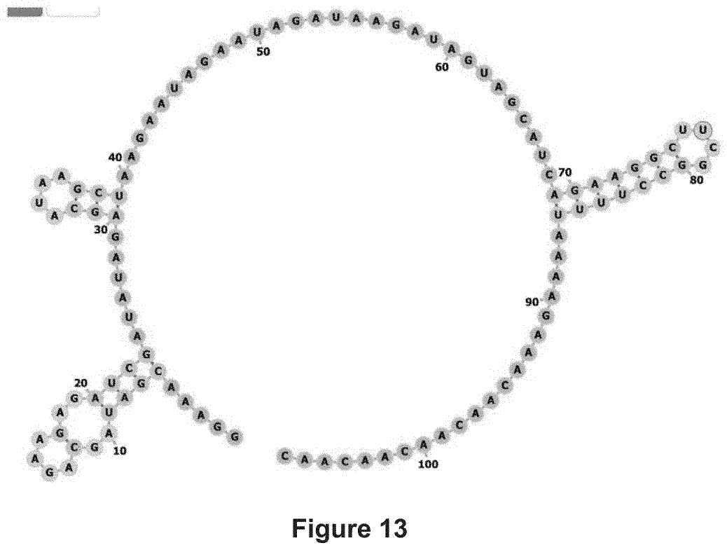

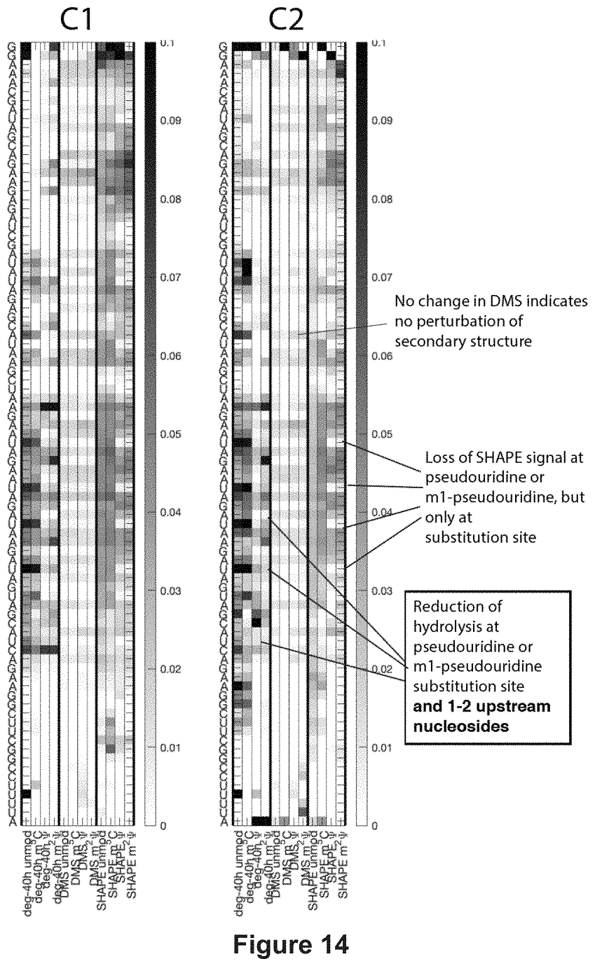

To show degradation in paired and unpaired regions, an exemplary RNA, C-1 (SEQ ID NO: 1172) was utilized which has the secondary structures illustrated in FIG. 13. As seen in FIG. 13, the RNA C-1 possesses both Watson-Crick pairs stems and unpaired loops. As such, degradation rates with single nucleotide resolution can be resolved using the RNA C-1 (SEQ ID NO: 1172). Turning to FIG. 14, the degradation results are illustrated of RNA C-1 (SEQ ID NO: 1172) and RNA C-2 (SEQ ID NO: 1173), showing that degradation is suppressed at the sites of modification of U to pseudouridine or 1-methyl-pseudouridine as well as 1-2 nucleotides 5′ to the sites of modification, consistent with local enhancement of base stacking. These data also show no effects of m5C on RNA structure or degradation in vitro, consistent with data measuring degradation rates of entire mRNAs (see FIG. 12).

The stabilization to in vitro degradation does not involve changes to global RNA structure. Experiments measuring chemical accessibility of the RNA to dimethyl sulfate (DMS) and 2′-hydroxyl acylating reagents (SHAPE), which are suppressed by formation of Watson-Crick pairs, show no change in structure; in particular regions that are unpaired in the two model RNAs remain accessible to both reagents. The only change seen is the SHAPE reactivity directly at the site of substitution of U to pseudouridine or 1-methyl-pseudouridine; this supports that 2′-hydroxyl chemical reactivity is locally decreased.

Conclusions: Overall, the data show that the mechanisms by which chemically modified nucleotides stabilize RNA degradation against hydrolysis in vitro are distinct from mechanisms by which such nucleotides change the properties of RNA in cells.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. An RNA therapeutic comprising:
an RNA molecule comprising a 5′ untranslated region, a 3′ untranslated region, and a coding sequence;
wherein the 5′ untranslated region is located 5′ of the coding sequence and the 3′ untranslated region is located 3′ of the coding sequence, and
wherein the coding sequence encodes for one or more viral epitopes.

2. The RNA therapeutic of claim 1, wherein the coding sequence is selected from the group consisting of: SEQ ID NO: 5 and SEQ ID NOs: 437-439.

3. The RNA therapeutic of claim 1, further comprising one or more of the group consisting of: a lubricant, a binder, a flavorant, and a coating.

4. The RNA therapeutic of claim 1, further comprising a capsule selected from the group consisting of: a virus, a viroid, a virion, a capsid, a bacterium, a lipid nanoparticle, a micelle, a DNA structure, and an RNA structure.

5. The RNA therapeutic of claim 1, wherein at least one nucleotide in the RNA molecule is replaced with an analog selected from the group consisting of: pseudouridine, 1-methyl-pseudouridine, and 5-methyl-cytidine, 1-methoxy-pseudouridine, and pseudo-isocytidine.

6. A method for increasing RNA stability comprising:
obtaining a target RNA sequence comprising a coding sequence;
altering at least one nucleotide within the RNA sequence, wherein the altered sequence improves a metric correlated with improved RNA function; and
synthesizing an RNA molecule representing the altered sequence.

7. The method of claim 6, wherein the altering step is performed by:
sampling a nucleotide within the target coding sequence, wherein the sampled nucleotide comprises an unpaired nucleotide within the coding sequence; and
substituting the sampled nucleotide with a new nucleotide to create a substituted coding sequence.

8. The method of claim 6, wherein the altered sequence possesses increased structure over the target coding sequence.

9. The method of claim 6, wherein the metric is selected from the group consisting of: free energy (dG) of an RNA molecule conformation, dG of the ensemble (dG(ensemble)), codon adaptation index (CAI), and expected Matthews Correlation Coefficient (MCC).

10. The method of claim 6, wherein the metric is selected from the group consisting of: maximum ladder distance (MLD), unpaired nucleotides, GC content, number of hairpins, number of 3-way junctions (3WJs), number of 4-way junctions, (4WJs), number of 5-way junctions (5WJs), ratios of hairpins to junctions, number of unpaired nucleotides, kissing loops, pseudoknots, tertiary contacts, multimeric designs, dimerization domains, and symmetrical structures.

11. The method of claim 6, wherein the metric is selected from the group consisting of: mean base pair proximity, probability of unpaired nucleotides, sum of paired bases, increased structure, summed probability of being unpaired, and predicted degradation score.

12. The method of claim 6, wherein the substituted coding sequence possesses a lower free energy than the target coding sequence.

13. The method of claim 6, wherein the target RNA sequence comprises at least one of the group consisting of: a poly-A tail, a 5′ untranslated region, and a 3′ untranslated region.

14. The method of claim 6, wherein the substituting step uses a greedy GC strategy, where if a C or G substitution is possible, the nucleotide is substituted for the nucleotide.

15. The method of claim 6, wherein the altered sequence possesses a lower DegScore than the target RNA sequence, wherein
DegScore=a*[stem nts]+b*[internal loop nts]+c*[hairpin nts]+d*[bulge nts]+e*[multiloop nts]+f*[exterior loop nts],
where nts stands for nucleotides, and
a-f represent coefficients for relative reactivity of nucleotides within a particular structure.

16. The method of claim 6, further comprising transfecting a cell with the synthesized RNA molecule.

17. The method of claim 6, further comprising treating an individual with the synthesized RNA molecule.

18. The method of claim 17, wherein the synthesized RNA molecule is formulated for medical use.

19. The method of claim 18, wherein the synthesized RNA molecule is formulated by combining the synthesized RNA molecule with at least one of the group consisting of: a lubricant, a binder, a flavorant, and a coating.

20. The method of claim 18, wherein the synthesized RNA molecule is encapsulated in at least one of the group consisting of: a virus, a viroid, a virion, a capsid, a bacterium, a lipid nanoparticle, a micelle, a DNA structure, and an RNA structure.

21. The method of claim 6, wherein altering at least one nucleotide within the RNA sequence comprises replacing at least one nucleotide in the RNA sequence with an analog selected from the group consisting of: pseudouridine, 1-methyl-pseudouridine, and 5-methyl-cytidine, 1-methoxy-pseudouridine, and pseudo-isocytidine.

22. The method of claim 6, wherein altering at least one nucleotide is iterated at least 100 times.

23. An RNA molecule to transfect a cell comprising: a 5′ untranslated region, a 3′ untranslated region, and a coding sequence, wherein the 5′ untranslated region is located 5′ of the coding sequence and the 3′ untranslated region is located 3′ of the coding sequence.

24. The RNA molecule of claim 23, wherein the coding sequence codes for one or more viral epitopes.

25. The RNA molecule of claim 24, wherein the coding sequence is selected from the group consisting of: SEQ ID NO: 5 and SEQ ID NOs: 437-439.

26. The RNA molecule of claim 23, wherein the coding sequence codes for green fluorescence protein.

27. The RNA molecule of claim 26, wherein the coding sequence is selected from the group consisting of: SEQ ID NO: 8 and SEQ ID NOs: 12-236.

28. The RNA molecule of claim 23, wherein the coding sequence codes for nanoluciferase.

29. The RNA molecule of claim 28, wherein the coding sequence is selected from the group consisting of SEQ ID NOs: 237-436.

30. The RNA molecule of claim 23, wherein at least one nucleotide in the RNA molecule is replaced with an analog selected from the group consisting of: pseudouridine, 1-methyl-pseudouridine, and 5-methyl-cytidine, 1-methoxy-pseudouridine, and pseudo-isocytidine.
